(19) United States
(12) Patent Application Publication
(10) Pub. No.: US 2025/0065008 A1
(43) Pub. Date: Feb. 27, 2025

(54) COMPOSITION CONTAINING MESENCHYMAL STEM CELLS AND HYDROGEL, AND USE THEREOF

(71) Applicant: JIANGYIN STEMEASY BIOTECH, LTD., Jiangyin, Jiangsu (CN)

(72) Inventors: Harry Huimin Chen, Jiangyin, Jiangsu (CN); Qifan Xu, Jiangyin, Jiangsu (CN); Haosheng Fei, Jiangyin, Jiangsu (CN)

(21) Appl. No.: 18/704,913

(22) PCT Filed: Oct. 26, 2022
(86) PCT No.: PCT/CN2022/127757

§ 371 Date: Nov. 11, 2024

(30) Foreign Application Priority Data
Oct. 26, 2021 (CN) 202111249937.9

(51) Int. Cl. A61L 24/00; C12N 5/0775

(52) U.S. Cl. CPC A61L 24/0031; A61L 24/0005; A61L 24/0015; A61L 24/0042; C12N 5/0668; A61L 2400/06; A61L 2430/36; C12N 2533/54; C12N 2533/72

(57) ABSTRACT

Provided are a composition containing mesenchymal stem cells and a hydrogel, and the use thereof. The mesenchymal stem cells are dispersed in the hydrogel. In the composition, the mesenchymal stem cells cooperate with the hydrogel, such that the healing of a fistula can be effectively promoted, the surgical operation difficulty and frequency are greatly reduced and the wound surface is also reduced, the discomfort of a patient in the perioperative period is alleviated, the disease course is shortened, the cure rate is increased, and the recurrence rate is reduced.

The present application claims the priority of Chinese patent application 2021112499379 filed on Oct. 26, 2021. The contents of the above Chinese patent application are incorporated herein by reference in their entireties.

### TECHNICAL FIELD

The present disclosure belongs to the field of stem cell tissue engineering, specifically relating to a composition comprising a mesenchymal stem cell and a hydrogel, and a use thereof.

### BACKGROUND

A fistula is an abnormal connection or passage between organs or blood vessels that are not usually connected, and it can form in different parts of the body. The causes of fistulas include trauma, surgery, medical complications, and diseases.

Anal fistula is a common and frequently occurring disease of the anorectum. Correctly locating and thoroughly sealing both the internal and external openings, reducing recurrence, and protecting sphincter function are key to the successful surgical treatment of anal fistulas. Protecting anal function is an important prerequisite for ensuring the postoperative quality of life of patients. Anal fistulas are often caused by infections around the anus, where local inflammation extensively damages the surrounding tissue and its stem cells, significantly reducing the tissue's repair capacity. Additionally, due to the large incisional drainage wounds from surgery, the pain is severe, and the postoperative wound healing is relatively slow. Traditional anal fistula surgeries also cause varying degrees of damage to the anal sphincter, leading to a high incidence of anal incontinence, which significantly decreases the patient's quality of life. Therefore, minimally invasive surgical techniques for the treatment of anal fistulas have become a focus of research among experts both domestically and internationally, giving rise to various minimally invasive procedures such as biological protein glue, anal fistula plugs, and stem cell therapy for anal fistulas. Although these minimally invasive surgeries have, to some extent, reduced surgical pain, improved success rates, and protected the anal sphincter, they still have a relatively high recurrence rate and various limitations.

Mesenchymal stem cells (MSCs) are a type of multipotent cell with self-renewing capabilities. Under specific induction conditions, both in vivo and in vitro, they can differentiate into various tissue cells such as adipocytes, osteocytes, chondrocytes, myocytes, tendons, ligaments, neurons, hepatocytes, cardiomyocytes, and endothelial cells. Even after continuous passaging and cryopreservation, they still retain their multipotent differentiation potential. In the medical community, they are referred to as universal cells. MSCs are easily sourced: they can be derived from bone marrow, adipose tissue, umbilical cord and placental tissue, peripheral blood, among others. Among these, umbilical cord mesenchymal stem cells (UCMSCs) derived from healthy umbilical cord tissue are particularly advantageous due to their ease of collection, lack of ethical controversy, large number of cells available, high vitality, ease of expansion and passaging, and strong immunomodulatory effects without issues of matching or rejection. This makes them the most promising multipotent stem cells for clinical application.

All hydrophilic or water-soluble polymers can form hydrogels through physical or chemical cross-linking. Hydrogels possess a grid-like three-dimensional structure and can absorb large amounts of water, swelling without dissolving in it. Scientists leverage this characteristic to combine stem cells with hydrogels for tissue repair. They believe hydrogels are an ideal medium for stem cell transplantation, aiding in the survival of stem cells within the body. There are a variety of hydrogel materials available for different purposes and applications, such as chitosan, sodium alginate, and gelatin. Similarly, depending on the specific case, hydrogel formulations can be modified to facilitate personalized treatment. Additionally, hydrogels, being biocompatible tissues, serve as a scaffold for cells and as a bridge connecting with tissues. They can also delay the degradation of stem cells and their secretions within the body, creating a sustained release system that maintains an effective concentration of stem cells for a prolonged period, thereby enhancing therapeutic outcomes. Research indicates that the therapeutic effects of stem cells in wound healing and tissue repair within physiological systems are primarily exerted through the secretion of exosomes. The three-dimensional network formed by hydrogels can mimic the normal microenvironment of the body, providing a rough surface conducive to cell adhesion, differentiation, and proliferation. Moreover, it can induce the paracrine system of stem cells, stimulating them to secrete exosomes, thereby enhancing their ability to repair and treat tissues. Collagen, as a well-established biomaterial in clinical applications, has long been widely used in tissue regeneration engineering due to its excellent biocompatibility, low immunogenicity, degradability, and safety.

Stem cells have enormous potential in treating various diseases. Adipose-derived stem cells (ADSCs), which have been isolated from adipose tissue in recent years, possess multipotent differentiation capabilities and have already seen extensive clinical application (Ceccarelli S, et al., “Immunomodulatory effect of adipose-derived stem cells: the cutting edge of clinical application.” Front Cell Dev Biol. 2020; 8:236). On Jan. 10, 2021, Takeda Pharmaceutical Company in Japan announced that it had submitted an application to the Ministry of Health, Labour and Welfare of Japan for the production and sale of Darvadstrocel (also known as Cx601). This product is intended for the treatment of complex perianal fistulas in adult patients with non-active or mildly active luminal Crohn's Disease (CD).

Currently, there are no related patents domestically, but clinical trials for ADSC treatment of Crohn's perianal fistula-related diseases have been conducted (Yang Zhang, et al., “Autologous adipose-derived stem cells for the treatment of complex cryptoglandular perianal fistula: a prospective case-control study.” Stem Cell Research & Therapy (2020) 11:475). In an article published by Zhou Chungen et al. in 2020, it was shown that compared to traditional incision and drainage treatment, ADSC is a feasible and effective method for treating Crohn's fistula. It protects anal function, reduces pain, enables faster recovery, has good tolerance, and improves the quality of life during the perioperative period.

In a 2021 article by Arthur Berger et al. (“Local administration of stem cell-derived extracellular vesicles in a thermoresponsive hydrogel promotes a pro-healing effect in a rat model of colo-cutaneous post-surgical fistula,” Nanoscale. 2021 Jan. 7; 13(1):218-232), it was demonstrated that extracellular vesicles (EVs) derived from stem cells and stromal cells (SCs), including exosomes, microvesicles, and vesicular bodies, are nanoscale (diameter 40-5000 nm) subcellular membrane-enclosed entities released constitutively or inducibly by cells. These EVs show promise in promoting tissue healing. Experiments have also confirmed the pleiotropic effects of EVs.

In a 2014 article by A. Beraru's team (“Efficacy of periurethral injections of polyacrylamide hydrogel (Bulkamid®) and quality of life of patients with urinary incontinence due to sphincter deficiency (IUE-IS).” Prog Urol. 2014 June; 24(8):501-10), a new treatment method using the invention of Bulkamid was applied to 80 women suffering from severe urinary incontinence due to sphincter deficiency. This involved periurethral injections of polyacrylamide hydrogel. During the follow-up period, with an average follow-up of 18.6±5.3 months per patient, 60% of the patients showed an improvement in their overall condition scores. There were no abscesses or infections at the injection sites, and no complications related to the product used. The hydrogel injections were found to be a safe and effective solution for female urinary incontinence, with the procedure being simple and complication-free, thereby improving the patients' quality of life.

In a 2021 article by E. Piantanida et al. (“Nanocomposite hyaluronic acid-based hydrogel for the treatment of esophageal fistulas.” Materials Today Bio Volume 10, March 2021, 100-109), an injectable nanocomposite hydrogel based on hyaluronic acid (HA) was developed to study its long-term healing promotion capabilities. HA is one of the most functionally diverse macromolecules in nature and is a crucial component of the natural extracellular matrix, playing a significant role in wound healing. The experiments confirmed that in situ injection of hyaluronic acid hydrogel is beneficial for treatment, offering convenient surgery and rapid recovery.

Based on the above information, it can be concluded that the use of stem cells/hydrogel for the treatment of anal fistulas offers several unique advantages, including minimal trauma, no sphincter damage, mild pain, rapid repair, low recurrence rate, and short hospital stays. The safety and efficacy have been preliminarily validated, and these advantages are not present in current surgical and minimally invasive traditional treatment methods.

Although Darvadstrocel is available abroad, the use of a single ADSC suspension for treating anal fistulas has a drawback: Darvadstrocel is only injected into the fistula wall and fistula opening without addressing the fistula tract, thereby not maximizing the utilization of stem cells. Additionally, domestic research in the field of stem cell treatment for anal fistulas has limitations such as a small number of cases, short follow-up periods, and insufficient postoperative evaluation. Moreover, the research direction heavily relies on autologous transplantation of ADSCs, which requires multiple surgeries, increasing patient discomfort. The limited availability of autologous stem cells also poses a risk of unusability.

### CONTENT OF THE PRESENT INVENTION

The technical problem to be solved by the present disclosure is to overcome the deficiencies in the existing technology, specifically the lack of an effective mesenchymal stem cell gel composition for treating fistulas. The present disclosure provides a composition comprising a mesenchymal stem cell and a hydrogel, and a use thereof. The composition of the present disclosure can be injected or filled into the fistula site, enhancing the therapeutic effect and reducing the difficulty and frequency of surgeries.

The present disclosure addresses the above technical problem through the following technical solutions:

In a first aspect, the present disclosure provides a composition comprising a mesenchymal stem cell and a hydrogel, wherein the mesenchymal stem cell is dispersed within the hydrogel.

In some embodiments of the present disclosure, the mesenchymal stem cell comprises one or more types of mesenchymal stem cells and/or a secretion thereof.

In some preferred embodiments of the present disclosure, the secretion is an extracellular vesicle.

In some further preferred embodiments of the present disclosure, the extracellular vesicle is selected from an exosome, a microvesicle, and a vesicular body.

In some embodiments of the present disclosure, the hydrogel comprises a gelling agent, and the gelling agent is selected from a natural gelling agent and a synthetic gelling agent.

In the present disclosure, the gelling agent can be classified into traditional hydrogels and responsive hydrogels based on external stimuli, including but not limited to, chemical-responsive types (such as pH), physical factor-responsive types (including temperature, light, electric field, magnetic field, sound field, pressure, etc.), and biological signal-responsive types (including enzymes, glucose, adenosine triphosphate, etc.).

In the present disclosure, the gelling agent can be classified based on the bonding mechanism into physical hydrogels (reversible gels) and chemical hydrogels (irreversible gels).

In some preferred embodiments of the present disclosure, the gelling agent is selected from one or more of the following: collagen, gelatin, hyaluronic gel, chitosan, hyaluronic acid, fibrin, alginic acid, cellulose, agarose, glucan, guar gum, proteins, ethylene glycol, acrylic acid and a derivative thereof, acrylamide and a derivative thereof, hydroxyethyl methacrylate and a derivative thereof, polyacrylic acid and a derivative thereof, and polymethacrylic acid and a derivative thereof.

In some further preferred embodiments of the present disclosure, the gelling agent is selected from one or more of the following: collagen (Col), methacrylated gelatin (GelMA) and a derivative thereof, methacrylated type I collagen and a derivative thereof, methacrylated type II collagen and a derivative thereof, methacrylated carboxymethyl chitosan (CMCSMA) and a derivative thereof, methacrylated type I alginate and a derivative thereof, methacrylated hyaluronic acid (HAMA) and a derivative thereof, methacrylated silk fibroin, and methacrylated heparin.

In one preferred embodiments of the present disclosure, the hydrogels in different formulations can be HAMA-based hydrogels, referred to as Hgel:

H1: HAMA 0.1%˜0.3%, GelMA 1%˜30%; H2: HAMA 0.3%˜0.5%, GelMA 1%˜30%; H3: HAMA 0.5%˜1%, GelMA 1%˜30%; H4: HAMA 1%˜2.5%, GelMA 1%˜30%. The hydrogels in different formulations can be CMCSMA-based hydrogels, referred to as Cgel:

C1: CMCSMA 0.25%˜0.5%, GelMA 1%˜30%; C2: CMCSMA 0.5%˜1%, GelMA 1%˜30%; C3: CMCSMA 1%˜2%, GelMA 1%˜30%; C4: CMCSMA2%˜5%, GelMA 1%˜30%.

The hydrogel preferably further comprises an additive, and the additive is selected from one or more of an initiator, a cross-linker, and an accelerator.

Herein, the initiator can be conventional in the art, preferably selected from one or more of photoinitiator 2959 (2-hydroxy-2-methyl-1-[4-(2-hydroxyethoxy)phenyl]-1-propanone), photoinitiator LAP (lithium phenyl-2,4,6-trimethylbenzoylphosphinate), and riboflavin.

The cross-linker can be conventional in the art, preferably N,N′-methylenebisacrylamide.

The accelerator can be conventional in the art, preferably tetramethylethylenediamine (TEMED).

In some embodiments of the present disclosure, the gelling agent is a combination of methacrylated gelatin and methacrylated hyaluronic acid, or a combination of methacrylated gelatin and methacrylated carboxymethyl chitosan.

In the present disclosure, the mesenchymal stem cell is derived from human umbilical cord tissue, human umbilical cord blood, human placenta, human adipose tissue, human bone marrow, human dental pulp, human menstrual blood, or mesenchymal-like stem cells derived from embryonic stem cells: the mesenchymal stem cell possesses multipotent differentiation potential and self-renewal capability.

Preferably, the mesenchymal stem cell is derived from human umbilical cord tissue, human umbilical cord blood, or human placenta.

The human umbilical cord mesenchymal stem cells (hUCMSC) used in the present disclosure are sourced from the embryonic umbilical cord, eliminating the need for multiple surgeries to obtain cells, which is free from ethical issues, and widely available, easy to collect, and easy to expand. They represent a more primitive MSC population with stronger proliferative capacity and more potent and diverse differentiation abilities. They exhibit lower expression of HLA-ABC (classical human leukocyte antigen class I antigens) and HLA-DR. They can also secrete factors such as GM-CSF and G-CSF, which other stem cells cannot secrete, promoting tissue regeneration.

The human umbilical cord mesenchymal stem cells (hUCMSC) of the present disclosure possess specific surface markers, including CD73+, CD90+, CD105+, CD34−, CD45−, CD14−, and CD19−, etc. (Dominici M, Le Blanc K, Mueller I, Slaper-Cortenbach I, Marini F, Krause D, Deans R, Keating A, Prockop Dj, Horwitz E. Minimal criteria for defining multipotent mesenchymal stromal cells. The International Society for Cellular Therapy position statement. Cytotherapy. 2006; 8(4):315-7).

In some embodiments of the present disclosure, the composition further comprises an auxiliary drug.

In one preferred embodiment of the present disclosure, the auxiliary drug is selected from one or more of immunosuppressants, analgesics, and anti-infective agents.

The immunosuppressants can alleviate the immune rejection response during allotransplantation. The immunosuppressants include, for example, mycophenolic acid drugs, glucocorticoids, calcineurin inhibitors, cyclosporine, tacrolimus, sirolimus, and everolimus. The analgesics can help treat inflammation or pain at the fistula site. The analgesics include, for example, non-steroidal drugs, opioid agonists, or salicylates.

The anti-infective agents can be used to prevent infection at the site treated with the composition. The anti-infective agents include, for example, antiparasitic drugs, anti-anaerobic drugs, aminoglycoside antibiotics, antifungal drugs, cephalosporin antibiotics, macrolide antibiotics, β-lactam antibiotics, penicillin antibiotics, quinolone antibiotics, sulfonamide antibiotics, and tetracycline antibiotics.

In a second aspect, the present disclosure provides a method for preparing the composition as described in the first aspect. The method comprises mixing the mesenchymal stem cell with the hydrogel in a vehicle to obtain the composition.

When the gelling agent of the hydrogel is collagen, the mixing temperature is 30-37.5° C.

When the gelling agent of the hydrogel is methacrylated gelatin and methacrylated hyaluronic acid, or methacrylated gelatin and methacrylated carboxymethyl chitosan: the condition for mixing is exposure to 365-405 nm light.

It should be understood by those skilled in the art that the vehicle is used to form a form of dispersed cells and does not affect cell growth or viability, and is non-toxic to the host. The vehicle is selected from compound electrolytes injection, physiological saline, PBS, and basal culture media.

The physiological saline can be a 0.85-0.9% sodium chloride aqueous solution, which is conventional in the art.

The basal culture media can be basal media conventionally used in the art for cell culture.

In one preferred embodiment of the present disclosure, the vehicle is compound electrolytes injection.

In the present disclosure, the vehicle is a pharmaceutically acceptable carrier, diluent, buffer, or other solvents known in the art. The vehicle should be sterile and capable of being produced, stored, and transported under stable conditions.

In a third aspect, the present disclosure provides a use of the composition as described in the first aspect for the preparation of a therapeutic agent for treating fistula.

In one preferred embodiment of the present disclosure, the fistula is selected from fistulas caused by Crohn's disease, autoimmune deficiency, injury, surgery, or infection.

In one further preferred embodiment of the present disclosure, the fistula is an anal fistula, for example, a complex anal fistula.

In some embodiments of the present disclosure, the complex anal fistula is a complex perianal fistula associated with non-active or mildly active luminal Crohn's disease; for example, it is used when the fistula is unresponsive to at least one conventional therapy or biological therapy and can only be used after the fistula has been repaired.

In one preferred embodiment of the present disclosure, the therapeutic agent is selected from regenerative tissue biopharmaceuticals, sprays, implants, or fillers.

In one further preferred embodiment of the present disclosure, the regenerative tissue biopharmaceutical is an injectable cell formulation.

In some embodiments of the present disclosure, the injectable cell formulation is an injectable cell suspension and/or an injectable cell gel formulation.

In a fourth aspect, the present disclosure provides a method for treating fistula, the method comprising injecting or filling the composition as described in the first aspect into the fistula site of a subject.

The preferred definition of the fistula is as described in the third aspect.

The injection or filling of the composition into the fistula site of the subject is carried out under general or local anesthesia in a surgical setting.

In one preferred embodiment of the present disclosure, the mesenchymal stem cell is formulated as a cell suspension of suitable concentration and volume, and are injected into tissues around one or more internal openings in the form of small vesicles using a syringe.

In another preferred embodiment of the present disclosure, the mesenchymal stem cells and hydrogel are mixed to prepare a cell gel formulation, which is injected into the lumen of the fistula using a syringe, and permitted to solidify at a temperature of 37° C. or through the application of blue light.

The act of injection herein refers to injecting the cell suspension or cell gel formulation into the fistula opening and fistula wall after debridement.

Filling the fistula tract with the stem cell gel allows for more extensive and comprehensive contact of the implant with the entire fistula tissue, not just limited to the internal fistula opening and fistula wall. After vigorous scraping with a tissue brush, the fistula epithelium is disrupted, and the composite implant is then filled in, forming continuity between the fistula walls. Collagen/gelling agents, being natural polymer materials, can support stem cell growth and possess certain tissue repair functions, thereby accelerating fistula tissue regeneration and healing.

In the present disclosure, natural gelling agents and/or synthetic gelling agents are added as auxiliary materials for stem cells. By leveraging the characteristics of collagen and photosensitive gelling agents to solidify at 37° C. or under 405 nm blue light, stem cells can be uniformly dispersed and solidified, avoiding issues such as uneven distribution, loss, or aggregation that can lead to changes in concentration and affect therapeutic outcomes. This approach maximizes the utilization of stem cells to achieve desired results, and adapts to the complex shapes of fistula tracts, thereby ensuring uniformity and reducing result deviations. Moreover, the solidification of collagen/photosensitive gelling agents after injection or filling allows them to better match the shape of complex fistula tracts, enhancing the convenience and stability of intracavity operations. With collagen/photosensitive gelling agents being important components of the extracellular matrix (ECM), the 3D local microenvironment constructed after solidification is more conducive to stem cell adhesion and growth. Furthermore, collagen/photosensitive gelling agents, being well-established biomaterials in clinical applications, have been widely used in tissue regeneration engineering. They can naturally degrade and promote tissue regeneration and repair.

In a fifth aspect, the present disclosure provides a use of the composition as described in the first aspect in the preparation of an in vivo microenvironment simulation system.

The in vivo microenvironment simulation system can be conventional in the art, for example, referring to a system that simulates the in vivo tissue environment derived from the same source as the samples through the interaction of various factors.

Based on common knowledge in the art, the above preferred conditions can be combined in any manner to obtain various preferred embodiments of the present disclosure.

The reagents and raw materials used in the present disclosure are all commercially available.

The positive advancements of the present disclosure are:
(1) The composition of the present disclosure, combining mesenchymal stem cells with hydrogel, can effectively promote fistula healing, achieving results comparable to or even better than traditional surgical methods. The composition significantly reduces the difficulty and frequency of surgeries, minimizes wound areas, reduces perioperative patient discomfort, shortens the course of the disease, improves cure rates, and lowers recurrence rates.
(2) The present disclosure uses hydrogel made from natural high molecular polymers, ensuring a good proliferation rate and viability rate of mesenchymal stem cells on the gel scaffold while maintaining the secretion of anti-inflammatory factors and exosomes. The use of mesenchymal stem cells addresses the ethical issues and source limitations associated with stem cell use, reduces the technical difficulty of the procedure, and avoids the pain caused by multiple surgeries for autologous stem cells.
(3) The collagen and photosensitive gelling agents used in the present disclosure can both promote the regulation of stem cell differentiation within the fistula microenvironment, foster tissue regeneration and healing at the fistula site, overcome the limitations of using single stem cells, enhance stem cell viability rates, prolong the retention and effectiveness of the composite implants at the treatment site, and reduce inflammatory stimulation of the local tissue caused by the composite implants.

### BRIEF DESCRIPTION OF THE DRAWINGS

FIG. 1 is a schematic diagram showing the results of Example 1.

FIG. 2 is a schematic diagram showing the proliferation rate results of Example 3.

FIG. 3 is a schematic diagram showing the migration rate results of Example 3.

FIG. 4 is a schematic diagram showing the healing time results of Example 6.

FIG. 5 is a schematic diagram showing the healing degree results of Example 6.

FIG. 6 is a schematic diagram showing the MRI results of Example 6;

In the figure: A represents pre-treatment, B represents the vehicle group, C represents the high-dose group, D represents the collagen+high-dose group, E represents the collagen+medium-dose group, and F represents the collagen+low-dose group.

FIG. 7 is a schematic diagram showing the histological section staining results of Example 6.

FIG. 8 is a schematic diagram showing the group results of histological section staining in Example 6.

In the figure: A represents day 1, B represents day 14, and C represents day 30.

FIG. 9 is a schematic diagram showing the transmission electron microscopy observation results of Example 2.

FIG. 10 is a schematic diagram showing the particle size detection results of Example 2.

FIG. 11 is a schematic diagram showing the surface marker identification results of Example 2.

FIG. 12 is a schematic diagram showing the retention and release experiment results of Example 4.

FIG. 13 is a schematic diagram showing the in vitro cell inflammation experiment results of Example 4.

FIG. 14 is a schematic diagram showing the fistula tract healing results of Example 8.

### DETAILED DESCRIPTION OF THE PREFERRED EMBODIMENT

The following examples further illustrate the present disclosure but do not limit the scope of the present disclosure to these specific embodiments. In the following examples, experimental methods without specified conditions are conducted according to conventional methods and conditions or selected based on the instructions provided in the product manuals.

TABLE 1
Biomaterials and reagents

| | | Catalog |
|---|---|---|
| | | number/ |
| | | batch |
| Name | Supplier | number |
| hUCMSC | SNC Stemcell | / |
| ADSC | Jiangyin Stemeasy | / |
| | Biotech Co., Ltd. | |
| Basal medium | HyClone | SH30023.01 |
| FBS | PAN | P30-3302 |
| Pancreatin | Gibco | 25200-072 |
| Penicillin-Streptomycin- | Procell | PB180121 |
| Amphotericin B solution | | |
| Collagen | Beijing Xiang-Zan | P01021 |
| | International Trading | |
| | Co., Ltd. | |
| CCK-8 kit | Shanghai Yeasen | 40203ES76 |
| | Biotechnology Co., Ltd. | |
| GelMA | Jiangyin Stemeasy | SE-3DP-0205 |
| | Biotech Co., Ltd. | |
| LAP (Lithium | Jiangyin Stemeasy | SE-3DP-0105 |
| phenyl-2,4,6-trimethyl- | Biotech Co., Ltd. | |
| benzoylphosphinate) | | |
| Rat | Wuxi Institute of | / |
| | Hematology Research and | |
| | Prevention | |
| TNBS (2,4,6- | SIGMA | P2297 |
| Trinitrobenzenesulfonic | | |
| acid) | | |
| Calcine-AM | Shanghai Yeasen | c6901160 |
| | Biotechnology Co., Ltd. | |
| Propidium iodide (PI) | Shanghai Yeasen | 40747ES76 |
| | Biotechnology Co., Ltd. | |
| Total RNA extraction | Solarbio | R1100 |
| reagent (Trizol) | | |
| Reverse transcription kit | Beyotime | AQ131-01 |
| Rat macrophage cell line | Wuhan Procell Life | CL-0190 |
| Raw264.7 | Science&Technology Co., | |
| | Ltd. | |
| Primers | Jiangyin Beibosi | Conventional |
| | Biotechnology Co., Ltd. | primer design |
| | | strategy |
| Lipopolysaccharide (LPS) | Solarbio | L8880 |
| Dexamethasone (Dex) | Solarbio | D8040 |

As known in the art, the complete medium is formed by adding FBS and Penicillin-Streptomycin-Amphotericin B solution to the basal medium.

### Example 1: Stem Cell Characterization of Mesenchymal Stem Cells Derived from Umbilical Cord Tissue

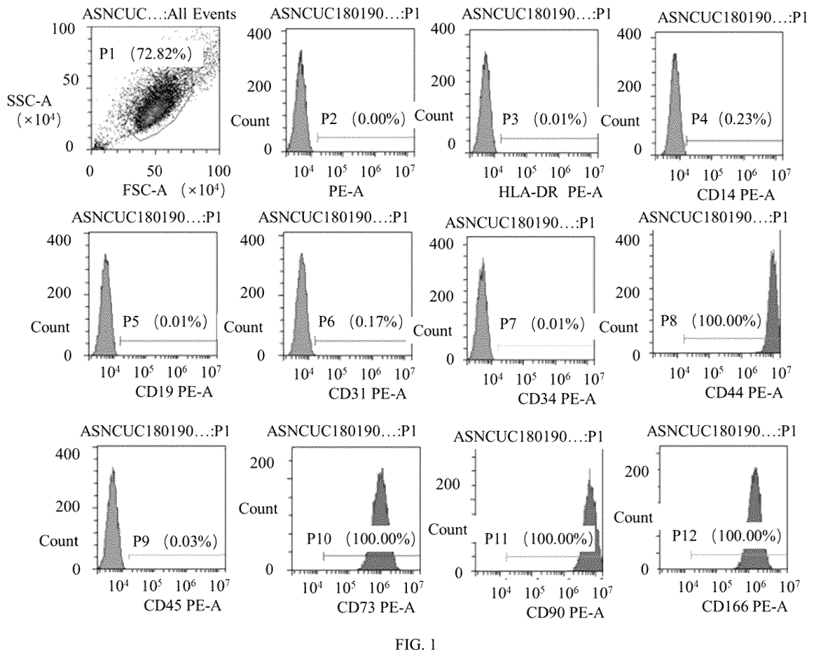

(1) The hUCMSC were cultured in a complete medium.
(2) The hUCMSC were subjected to cell characterization for HLA-DR, CD14, CD19, CD31, CD34, CD44, CD45, CD73, CD90, and CD166, with the results shown in FIG. 1.

As illustrated in FIG. 1, the cultured hUCMSC cells exhibited negative expression rates for HLA-DR, CD14, CD19, CD31, CD34, and CD45, while positive expression rates were observed for CD44, CD73, CD90, and CD166, which is consistent with the characteristics of umbilical cord mesenchymal stem cells.

### Example 2: Identification of Exosomes from Mesenchymal Stem Cells Derived from Umbilical Cord Tissue

### 1. Isolation and Identification of Exosomes

The supernatant from the hUCMSC in Example 1 was subjected to exosome extraction and identification using an exosome extraction kit, following the methodology described in the literature (Xiao Li et al., “Isolation and Identification of Exosomes from Umbilical Cord Mesenchymal Stem Cells,” Chinese Journal of Cell and Stem Cell Research: Electronic Edition, 2016, Issue 4, pp. 236-239). The sterile exosome suspension obtained was stored in a −80° C. refrigerator for future use.

### 2. Identification Results:

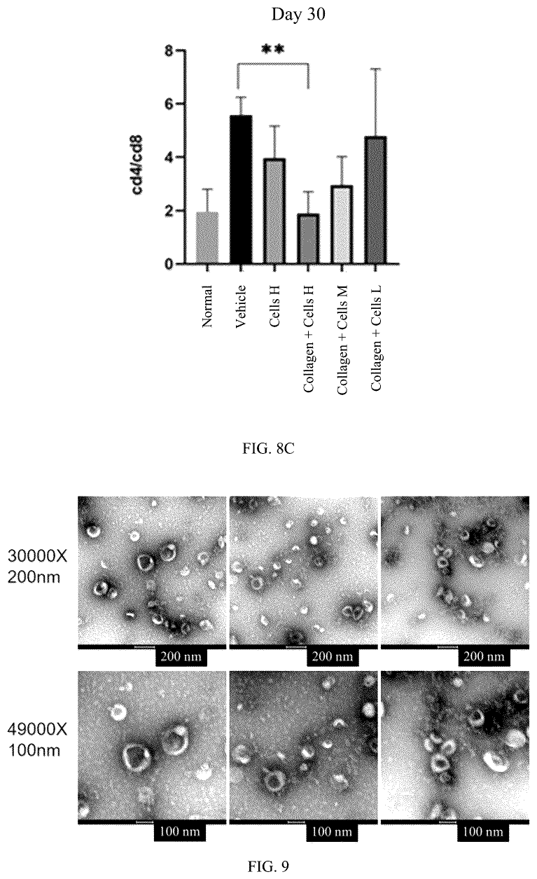

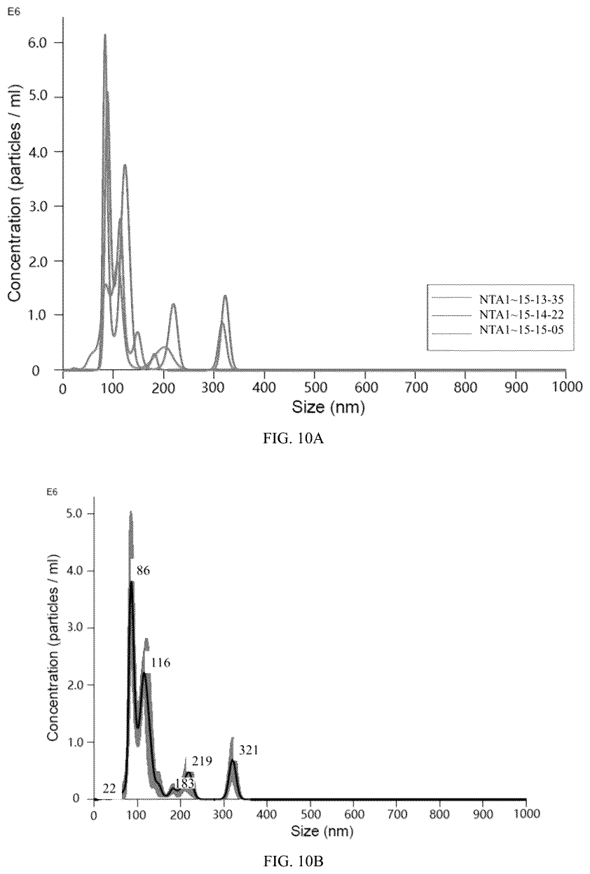

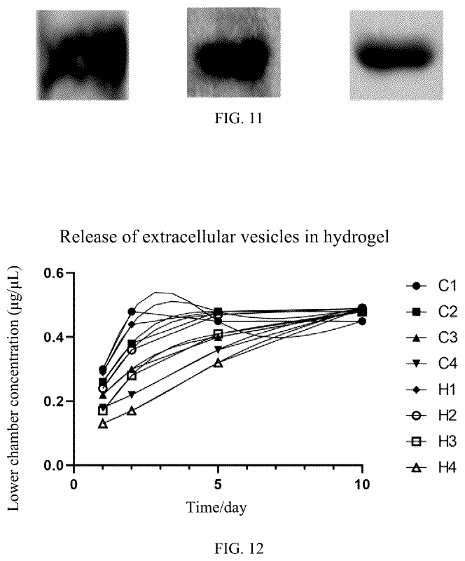

(1) Morphology was observed using transmission electron microscopy, as shown in FIG. 9.
(2) Particle size was detected using a nanoparticle tracking analyzer (NTA), as shown in FIG. 10.
(3) Surface markers CD63, CD81, and Tsg101 were identified, as shown in FIG. 11.

The identification results indicated that the obtained exosomes conformed to the characteristics of extracellular vesicles.

### Example 3: Preparation of Collagen Solution (Agent A-1)

(1) Sedimentation rate and viability rate: Collagen was dissolved in acetic acid at concentrations of 10, 7.5, 5, 2.5, and 1 mg/mL. The pH was adjusted to 7.0 using sodium hydroxide, and the volume was supplemented with basal culture medium. Appropriate concentrations of ADSC were then added for sedimentation and culture experiments. Cell viability and mortality were observed using calcine-AM/PI double staining, with results shown in Table 2.

TABLE 2
Collagen test results

| Collagen concentration | | |
|---|---|---|
| (mg/mL) | Sedimentation test | Cultivation test |
| 1 | Complete sedimentation | 100% viability |
| 2.5 | Slight sedimentation | >95% viability |
| 5 | No sedimentation | >90% viability |
| 7.5 | No sedimentation | About 50% viability |

| | |
|---|---|
| 10 | Concentration too high to dissolve |

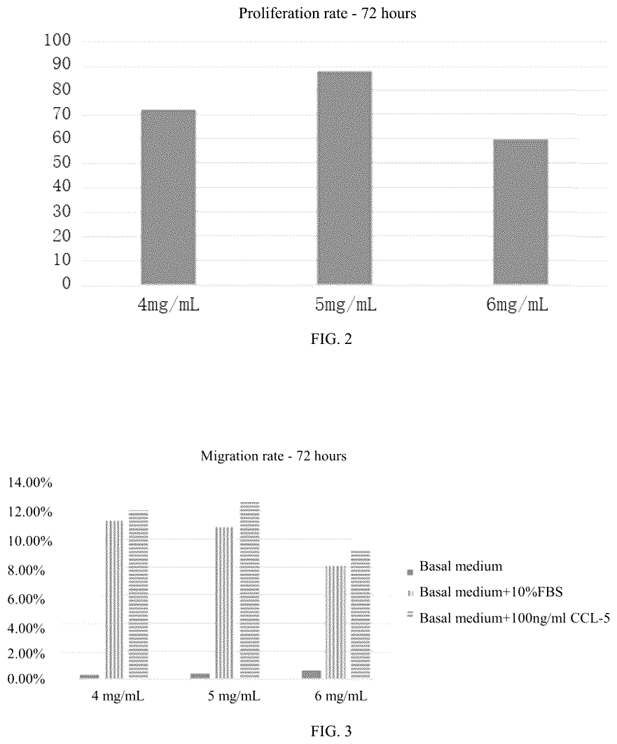

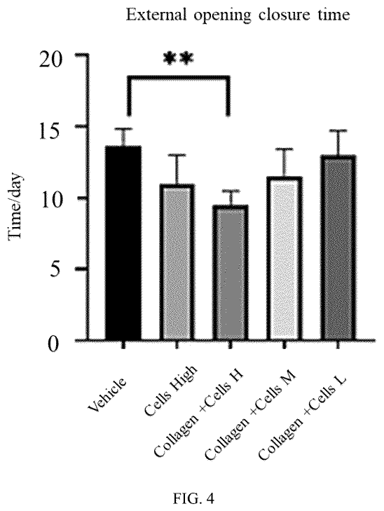

(2) Proliferation rate test: A suitable amount of ADSC cells was taken, centrifuged, and then mixed with the prepared Agent A-1. The mixture was placed into a 96-well plate and cultured for 1, 3, 7, 14, and 21 days respectively. At the specified time points, a CCK-8 staining assay was performed. OD450 measurements were taken and corresponding standard curves were used to calculate the cell number, followed by the calculation of the proliferation rate. The results are shown in FIG. 2.
(3) Migration rate: A suitable amount of ADSC cells was taken, centrifuged, and then mixed with the prepared Agent A-1. The mixture was then added to a Transwell chamber, with the lower chamber containing basal medium, basal medium with 10% FBS, or basal medium with 100 ng/mL CCL-5, respectively. The migration time was set to 72 hours. At the specified time points, a CCK-8 staining assay was performed. OD450 measurements were taken and corresponding standard curves were used to calculate the cell number, followed by the calculation of the migration rate. The results are shown in FIG. 3.

Results: Collagen concentrations ranging from 1 to 7.5 mg/mL demonstrated acceptable sedimentation rates, viability rates, migration rates, and proliferation rates, fulfilling the experimental requirements of minimal sedimentation, >90% viability, and observable proliferation and migration. A collagen concentration of approximately 5 mg/mL exhibited the best outcomes for viability and migration rates.

### Example 4: Preparation of Hydrogels (Agent A-2 and Agent A-3)

(1) Methacrylated gelatin and methacrylated hyaluronic acid were diluted with a vehicle (basal medium containing 0.1% (m/v) LAP) to the concentrations specified in Table 3, resulting in Agent A-2. Methacrylated gelatin and methacrylated carboxymethyl chitosan were diluted with a vehicle (basal medium containing 0.1% (m/v) LAP) to the concentrations specified in Table 4, resulting in Agent A-3.
(2) The sedimentation rate and viability rate of hUCMSCs in the hydrogel were the same as those observed in Example 3.

TABLE 3
hUCMSC sedimentation and viability
test results in hydrogel agent A-2

| HAMA | GelMA | Sedimentation rate | Viability rate |
|---|---|---|---|
| <0.1% | <1% | Unable to form gel | 100% viability |
| 0.1%~2.5% | 1%~30% | No sedimentation | >95% viability |
| >2.5% | >30% | Unable to dissolve | — |

TABLE 4
hUCMSC sedimentation and viability
test results in hydrogel agent A-3

| CMCSMA | GelMA | Sedimentation test | Viability rate |
|---|---|---|---|
| <0.25% | <1.5% | Unable to form gel | 100% viability |
| 0.25%~5% | 1.5%~30% | No sedimentation | >95% viability |
| >5% | >30% | Concentration too high | — |

(3) Retention and release of umbilical cord-derived mesenchymal stem cell exosomes in hydrogels

Exosomes from Example 2 were mixed with Agent A-2 and Agent A-3 to prepare different formulations of hydrogels with an extracellular vesicle concentration of 0.5 μg/μL. Hydrogel with HAMA as the main component is referred to as Hgel, H1: HAMA 0.1%˜0.3%, GelMA 1%˜30%; H2: HAMA 0.3%˜0.5%, GelMA 1%˜30%; H3: HAMA 0.5%˜1%, GelMA 1%˜30%; H4: HAMA 1%˜2.5%, GelMA 1%˜30%. Hydrogel with CMCSMA as the main component is referred to as Cgel, C1: CMCSMA 0.25%˜0.5%, GelMA 1%˜30%; C2: CMCSMA 0.5%˜1%, GelMA 1%˜30%; C3: CMCSMA 1%˜2%, GelMA 1%˜30%; C4: CMCSMA 2%˜5%, GelMA 1%˜30%. These hydrogel formulations were placed in the upper chamber of a Transwell chamber, with the lower chamber containing the basal medium. Samples were taken on Days 1, 2, 5, and 10 to measure the concentration of extracellular vesicles released into the lower chamber using the BCA protein assay method.

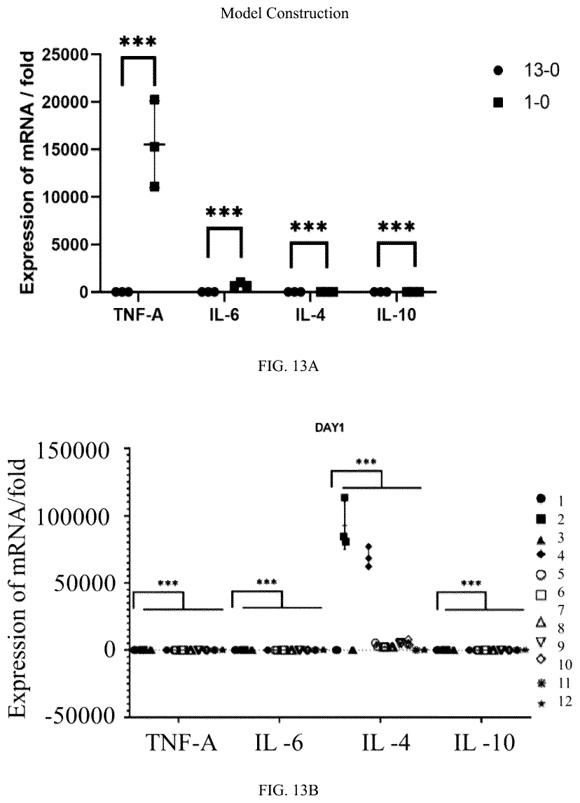

The results, as shown in FIG. 12, indicate that extracellular vesicles can be retained within the hydrogel for more than 2 days.
(4) In vitro cell inflammation assay of umbilical cord-derived mesenchymal stem cell exosomes

Rat macrophage cell line Raw264.7 cells were seeded at a density of 1×10⁵ cells/mL in six-well plates. After stable adhesion, cells were stimulated and treated according to the conditions outlined in Table 5. For each group, mRNA levels of TNF-α, IL-6, IL-4, and IL-10 were analyzed using RT-qPCR on Days 1, 3, and 7.

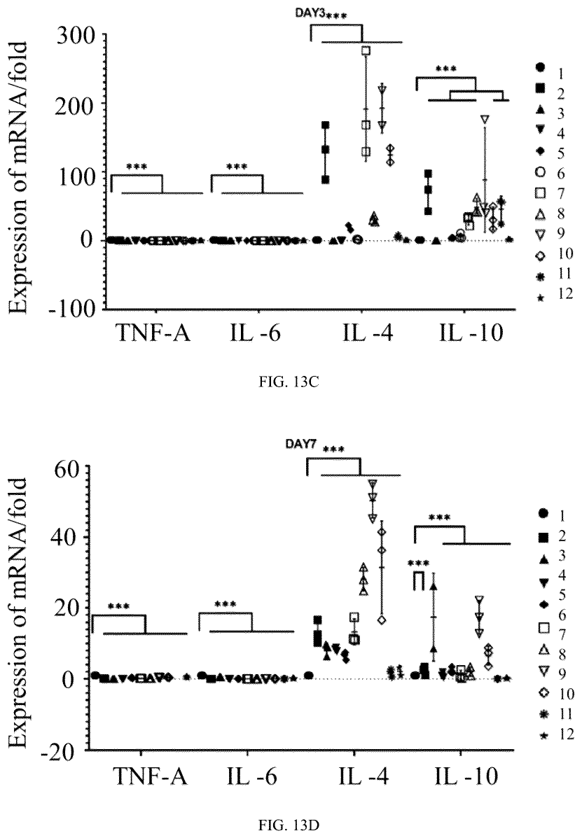

The results, shown in FIG. 13, indicate that all data points exhibit significant differences. The combination of hydrogels with stem cells/extracellular vesicles significantly enhanced the expression of the IL-4 factor and effectively inhibited cellular inflammation.

TABLE 5

| Group | Treatment |
|---|---|
| No. | After 12 hours of stimulation with 100 ng/mL LPS, the medium |
| 1 | was replaced with basal medium, with medium changes every |
| | 3 days. |
| No. | After 12 hours of stimulation with 100 ng/mL LPS, the medium |
| 2 | was replaced with basal medium containing 1 μg/mL Dex, with |
| | medium changes every 7 days. |
| No. | After 12 hours of stimulation with 100 ng/mL LPS, the medium |
| 3 | was replaced with blank Cgel and basal medium, with medium |
| | changes every 3 days. |
| No. | After 12 hours of stimulation with 100 ng/mL LPS, the medium |
| 4 | was replaced with blank Hgel and basal medium, with medium |
| | changes every 3 days. |
| No. | After 12 hours of stimulation with 100 ng/mL LPS, the medium |
| 5 | was replaced with basal medium containing 1 μg/μL |
| | exosomes, with medium changes every 3 days. |
| No. | After 12 hours of stimulation with 100 ng/mL LPS, the medium |
| 6 | was replaced with basal medium containing 0.5 μg/μL |
| | exosomes, with medium changes every 3 days. |
| No. | After 12 hours of stimulation with 100 ng/mL LPS, the medium |
| 7 | was replaced with blank Cgel (hUCMSC 5 × 10⁶ cells/mL) and |
| | basal medium, with medium changes every 3 days. |
| No. | After 12 hours of stimulation with 100 ng/mL LPS, the medium |
| 8 | was replaced with blank Hgel (hUCMSC 5 × 10⁶ cells/mL) and |
| | basal medium, with medium changes every 3 days. |
| No. | After 12 hours of stimulation with 100 ng/mL LPS, the medium |
| 9 | was replaced with blank Cgel (1 μg/μL exosomes) and basal |
| | medium, with medium changes every 3 days. |
| No. | After 12 hours of stimulation with 100 ng/mL LPS, the medium |
| 10 | was replaced with blank Hgel (1 μg/μL exosomes) and basal |
| | medium, with medium changes every 3 days. |
| No. | After 12 hours of stimulation with 100 ng/mL LPS, the medium |
| 11 | was replaced with blank Cgel (0.5 μg/μL exosomes) and basal |
| | medium, with medium changes every 3 days. |
| No. | After 12 hours of stimulation with 100 ng/mL LPS, the medium |
| 12 | was replaced with blank Hgel (0.5 μg/μL exosomes) and basal |
| | medium, with medium changes every 3 days. |
| No. | Without LPS stimulation, the cells were maintained in basal |
| 13 | medium throughout, with medium changes every 3 days. |

Note:
Hgel and Cgel refer to the H4 and C4 formulations from the retention experiment in Example 4 (3), respectively.

### Example 5: Preparation of Stem Cell Suspension (Agent B-1) and Extracellular Vesicle Suspension (Agent B-2) for Treating Fistulas

The hUCMSCs cultured in Example 1 were digested and centrifuged, then diluted with a vehicle (basal medium) to obtain a stem cell suspension. The suspension was prepared in three different concentrations for in vivo experiments: high dose (5×10⁶ cells/mL), medium dose (1×10⁶ cells/mL), and low dose (0.2×10⁶ cells/mL).

Extracellular vesicles obtained in Example 2 were diluted with a vehicle to prepare an extracellular vesicle suspension, which was also prepared in various concentrations for in vivo experiments.

### Example 6: Effects of Umbilical Cord-Derived Mesenchymal Stem Cell and Collagen Formulation on Recovery of Anal Fistula Model

The collagen solution Agent A-1 and the stem cell suspension Agent B-1 were used in this example.

The experimental procedure was divided into three stages:
(1) Rat modeling: Rats were modeled following the method described by Meredith Flacs, MD, Maxime Collard, MD, Sabrina Doblas, PhD, Magaly Zappa, MD, PhD, Dominique Cazals-Hatem, MD, Léon Maggiori, MD, PhD, Yves Panis, MD, PhD, Xavier Treton MD, PhD, Eric Ogier-Denis, PhD. Preclinical Model of Perianal Fistulizing Crohn's Disease. Original Research Article-Basic Science, which involves TNBS enema (to induce colitis) and anal perforation (to induce anal fistula).
(2) Treatment: The rats were divided into five groups, including one control group, i.e., the vehicle group (3 subjects). The experimental groups consisted of four groups: the high-dose stem cell group (5×10⁶ cells/mL) (denoted as “Cells H” or “Cells High” in the figures) with 3 subjects: the 5 mg/mL collagen+high-dose stem cell group (5×10⁶ cells/mL) (denoted as “Collagen+Cells H” in the figures) with 4 subjects; the 5 mg/mL collagen+medium-dose stem cell group (1×10⁶ cells/mL) (denoted as “Collagen+Cells M” in the figures) with 4 subjects; and the 5 mg/mL collagen+low-dose stem cell group (0.2×10⁶ cells/mL) (denoted as “Collagen+Cells L” in the figures) with 3 subjects. The treatment of anal fistula was conducted according to the methods documented in Tihomir Georgiev Hristov & H. Guadalajara & M. D. Herreros & A. L. Lightner & E. J. Dozois & M. García-Arranz & D. García-Olmo. A Step-By-Step Surgical Protocol for the Treatment of Perianal Fistula with Adipose-Derived Mesenchymal Stem Cells. Journal of Gastrointestinal Surgery.
(3) Efficacy evaluation:
a. Peripheral blood flow cytometry immunoassay: Conducted on Days 1, 14, and 30 post-treatment.
b. MRI imaging: Conducted pre-treatment and on Day 42 post-treatment.
c. Histological section staining: Conducted at the endpoint on Day 45 post-treatment.

### Results:

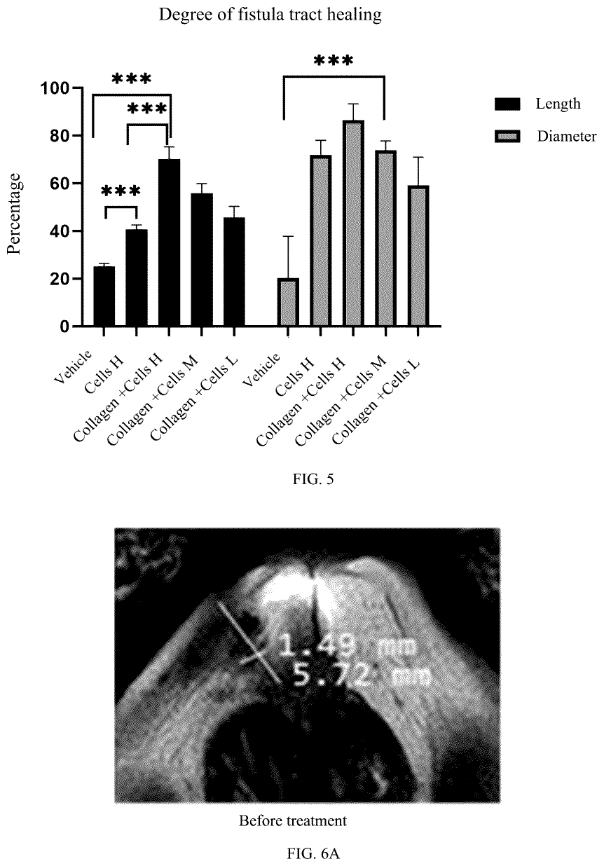

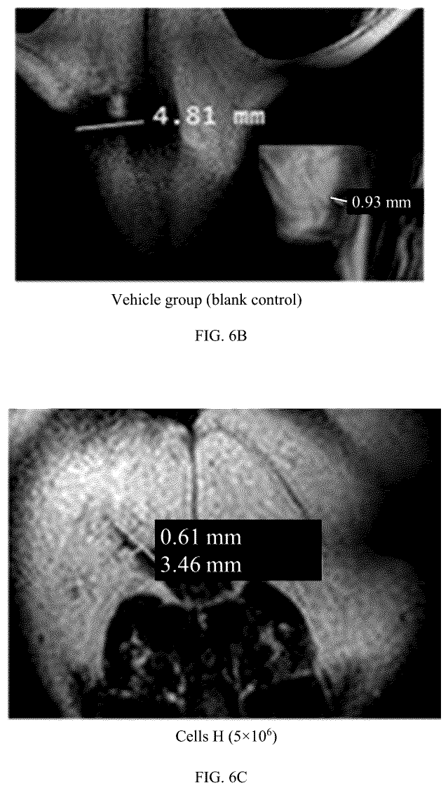

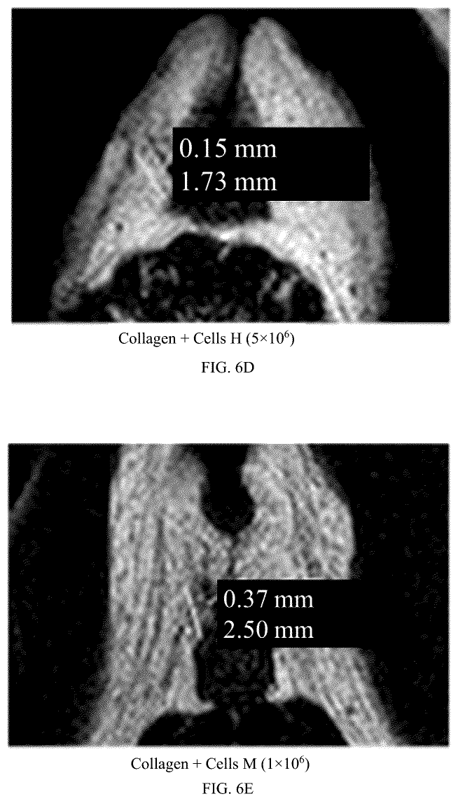

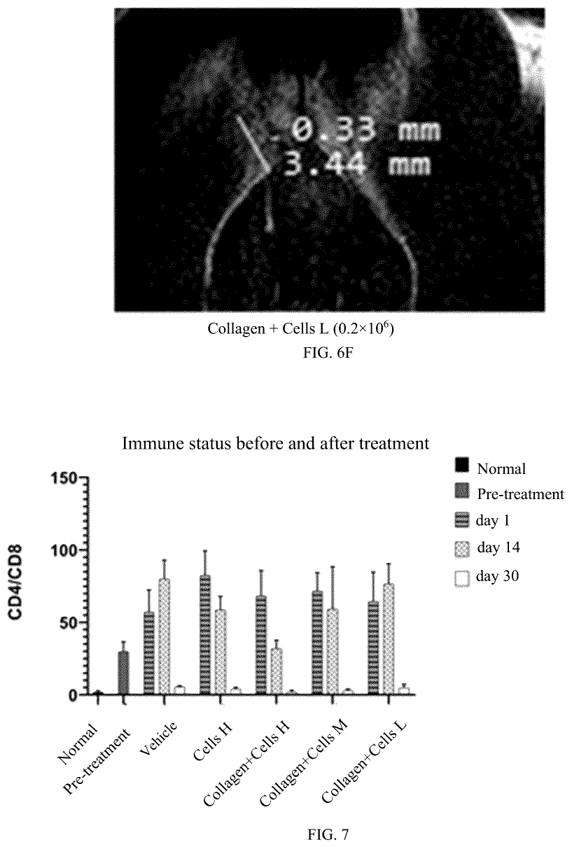

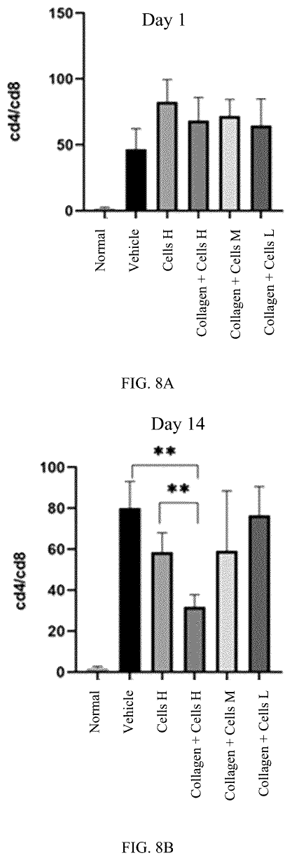

1) The healing time of external openings in the experimental groups was shorter than in the control group, with the high-concentration cell group showing better results than the low-concentration group. The groups treated with collagen showed better results than those without collagen. The group treated with 5 mg/mL collagen and high-dose stem cells (5×10⁶ cells/mL) showed a significant difference compared to the control group, as shown in FIG. 4.
2) Regarding fistula tract healing detected by MRI, the degree of fistula tract healing in the experimental groups was better than in the control group, with the high-concentration cell group showing better results than the low-concentration group. The groups treated with collagen showed better results than those without collagen, with significant differences observed, as shown in FIGS. 5 and 6.
(3) Regarding peripheral immune conditions, the immune recovery in the experimental groups was better than in the control group, with the high-concentration cell group showing better results than the low-concentration group. The groups treated with collagen showed better results than those without collagen, with significant differences observed between groups, as shown in FIGS. 7 and 8.

It is well-known for those skilled in the art that when the effects of the hydrogel on cell viability and migration in vitro are understood, it can be reasonably expected that the hydrogel will have similar effects on stem cells in vivo.

### Example 7: Effects of Umbilical Cord-Derived Mesenchymal Stem Cell Composite Hydrogel on Recovery of In Vivo Anal Fistula Model

The hydrogel Agent A-2 and stem cell suspension Agent B-1 were used in this example.

The experimental procedure follows the steps outlined in Example 6.

### Example 8: Effects of Umbilical Cord-Derived Mesenchymal Stem Cell Extracellular Vesicle Composite Hydrogel on Recovery of In Vivo Anal Fistula Model

The hydrogel Agent A-2 and extracellular vesicle suspension Agent B-2 were used in this example.

The experimental procedure follows the steps outlined in Example 6. Examples 7 and 8 were divided into six groups, including one control group, i.e., the vehicle group (4 subjects). The experimental groups consisted of four groups: the 5 mg/mL collagen+high-dose stem cell group (5×10⁶ cells/mL) (denoted as “Col+hUCMSC” in the figures) with 4 subjects: the hydrogel+high-dose stem cell group (5×10⁶ cells/mL) (denoted as “Hgel+hUCMSC” in the figures) with 4 subjects: the hydrogel+high-dose extracellular vesicles group (equivalent to the extract from 1×10⁷ hUCMSC cells) (denoted as “Hgel+EV-H” in the figures) with 4 subjects: the hydrogel+medium-dose extracellular vesicles group (equivalent to the extract from 5×10⁶ hUCMSC cells) (denoted as “Hgel+EV-M” in the figures) with 4 subjects; and the hydrogel+low-dose extracellular vesicles group (equivalent to the extract from 2×10⁶ hUCMSC cells) (denoted as “Hgel+EV-L” in the figures) with 4 subjects.

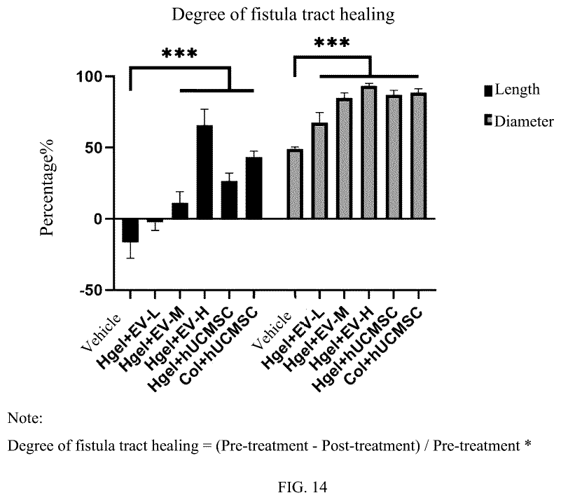

The degree of fistula tract healing is shown in FIG. 14. The results indicate that the combination of hydrogel and stem cells/extracellular vesicles is more effective than the control group (surgical suturing).

Although the specific embodiments of the present invention have been described above, it should be understood by those skilled in the art that these are merely illustrative examples. Various changes or modifications can be made to these embodiments without departing from the principles and spirit of the present invention. Therefore, the scope of protection of the present disclosure is defined by the appended claims.

1. A composition comprising a mesenchymal stem cell and a hydrogel, wherein the mesenchymal stem cell is dispersed within the hydrogel.

2. The composition according to claim 1, wherein the mesenchymal stem cell comprises one or more types of mesenchymal stem cells and/or a secretion thereof;
preferably, the secretion is an extracellular vesicle;
more preferably, the extracellular vesicle is selected from an exosome, a microvesicle, and a vesicular body.

3. The composition according to claim 1, wherein the hydrogel comprises a gelling agent, and the gelling agent is selected from a natural gelling agent and a synthetic gelling agent;
preferably, the gelling agent is selected from one or more of the following: collagen, gelatin, hyaluronic gel, chitosan, hyaluronic acid, fibrin, alginic acid, cellulose, agarose, glucan, guar gum, proteins, ethylene glycol, acrylic acid and a derivative thereof, acrylamide and a derivative thereof, hydroxyethyl methacrylate and a derivative thereof, polyacrylic acid and a derivative thereof, and polymethacrylic acid and a derivative thereof;
more preferably, the gelling agent is selected from one or more of the following: collagen, methacrylated gelatin and a derivative thereof, methacrylated type I collagen and a derivative thereof, methacrylated type II collagen and a derivative thereof, methacrylated carboxymethyl chitosan and a derivative thereof, methacrylated type I alginate and a derivative thereof, methacrylated hyaluronic acid and a derivative thereof, methacrylated silk fibroin, and methacrylated heparin.

4. The composition according to claim 3, wherein the hydrogel further comprises an additive, and the additive is selected from one or more of an initiator, a cross-linker, and an accelerator;
the initiator is preferably selected from one or more of photoinitiator 2959 (2-hydroxy-2-methyl-1-[4-(2-hydroxyethoxy)phenyl]-1-propanone), photoinitiator LAP (lithium phenyl-2,4,6-trimethylbenzoylphosphinate), and riboflavin;
the cross-linker is preferably N,N′-methylenebisacrylamide; the accelerator is preferably tetramethylethylenediamine.

5. The composition according to claim 3, wherein the gelling agent is a combination of methacrylated gelatin and methacrylated hyaluronic acid, or a combination of methacrylated gelatin and methacrylated carboxymethyl chitosan.

6. The composition according to claim 1, wherein the mesenchymal stem cell is derived from human umbilical cord tissue, human umbilical cord blood, human placenta, human adipose tissue, human bone marrow, human dental pulp, human menstrual blood, or mesenchymal-like stem cells derived from embryonic stem cells; the mesenchymal stem cell possesses multipotent differentiation potential and self-renewal capability;
the mesenchymal stem cell is preferably derived from human umbilical cord tissue, human umbilical cord blood, or human placenta.

7. The composition according to claim 1, wherein the composition further comprises an auxiliary drug;
preferably, the auxiliary drug is selected from one or more of immunosuppressants, analgesics, and anti-infective agents.

8. A method for preparing the composition according to claim 1, wherein the method comprises:
mixing the mesenchymal stem cell with the hydrogel in a vehicle to obtain the composition; preferably:
when the gelling agent of the hydrogel is collagen, the mixing temperature is 30-37.5° C.;
when the gelling agent of the hydrogel is methacrylated gelatin and methacrylated hyaluronic acid, or methacrylated gelatin and methacrylated carboxymethyl chitosan; the condition for mixing is exposure to 365-405 nm light.

9. The method according to claim 8, wherein the vehicle is used to form a form of dispersed cells and does not affect cell growth or viability, and is non-toxic to a host; the vehicle is selected from one or more of compound electrolytes injection, physiological saline, PBS, and basal culture media;
preferably, the vehicle is compound electrolytes injection.

10. A therapeutic agent for treating fistula, comprising the composition according to claim 1;
preferably, the fistula is selected from fistulas caused by Crohn's disease, autoimmune deficiency, injury, surgery, or infection;
more preferably, the fistula is an anal fistula, for example, a complex anal fistula;
further more preferably, the complex anal fistula is a complex perianal fistula associated with non-active or mildly active luminal Crohn's disease.

11. The therapeutic agent according to claim 10, wherein the therapeutic agent is selected from one or more of regenerative tissue biopharmaceuticals, sprays, implants, and fillers;
preferably, the regenerative tissue biopharmaceutical is an injectable cell formulation;
more preferably, the injectable cell formulation is an injectable cell suspension and/or an injectable cell gel formulation.

12. A method for treating fistulas in a subject in need thereof, comprising: administering an effective amount of the composition according to claim 1 to the subject.

13. A method for simulating microenvironment in vivo, comprising culturing a cell with the composition according to claim 1.
